(19) United States
(12) Patent Application Publication
(10) Pub. No.: US 2018/0177404 A1
(43) Pub. Date: Jun. 28, 2018

(54) Gradient Index Lens Assembly-Based Imaging Apparatus, Systems and Methods

(71) Applicant: LightLab Imaging, Inc., Westford, MA (US)

(72) Inventor: Yu Liu, Irvine, CA (US)

(73) Assignee: LightLab Imaging, Inc., Westford, MA (US)

(21) Appl. No.: 15/736,575

(22) PCT Filed: Jun. 23, 2016
(86) PCT No.: PCT/US2016/039019

§ 371 Date: Dec. 14, 2017

Related U.S. Application Data
(60) Provisional application No. 62/185,280, filed on Jun. 26, 2015.

(51) Int. Cl. A61B 5/00; G02B 6/42

(52) U.S. Cl. CPC A61B 5/0084; A61B 5/0066; A61B 3/102; G02B 6/4212; G02B 6/4206

(57) ABSTRACT

In part, the disclosure relates to a lens assembly. The lens assembly can be used to direct light for sensing and imaging. In one embodiment, the lens assembly is a component of an intravascular data collection probe such as an optical coherence tomography probe. The lens assembly can include an optical fiber having a first diameter and a gradient index lens that includes a rod having a length L. The rod can include a substantial planar end and a polished end. The rod can include a longitudinal axis and a second diameter. The second diameter is greater than the first diameter in one embodiment. The substantially planar end is optically coupled to an endface of the optical fiber. The refractive index changes along the length L of the rod.

### RELATED APPLICATIONS

This application claims priority to and the benefit of U.S. Provisional Patent Application No. 62/185,280 filed on Jun. 26, 2015, the disclosure of which is herein incorporated by reference in its entirety.

### FIELD OF THE INVENTION

The technical field of the disclosure relates generally to optical elements, the design and manufacture of optical elements, and methods of using the same. In addition, the disclosure also relates to using optical elements to collect data with respect to a sample of interest.

### BACKGROUND

Medical diagnostic techniques which rely on measuring the optical properties of a narrow, twisting lumen (e.g., small arteries and veins) or a small space (e.g., pulmonary airways) require small optical probes. These probes in turn require small imaging lenses and associated scanning and beam director elements.

Optical analysis methods such as interferometric methods deliver light onto a sample of interest, and further require collection of a portion of the light returned from the sample. Due to the size and complexity of many light sources and light analysis devices, they are typically located remotely from the sample of interest. This is especially apparent when the sample of interest is an internal part of a larger object, such as biological tissue inside of a living organism. One method of optically analyzing blood vessels, tissue, and other internal body parts and systems is to guide light from a remote light source onto the sample using a thin optical fiber. An example of such a method is the optical analysis of a luminal organ, such as a blood vessel, using a fiber-optic catheter that is connected on one end to a light source outside of the body while the other end is inserted into the vessel.

A significant barrier to conducting optical analysis of internal regions, such as lumens, is the design and low-cost manufacture of miniature optical devices for focusing or collimating light. Many types of optical analysis, such as imaging and spectroscopy, require that the light incident on the sample be focused at a particular distance or substantially collimated. Since light radiating from the tip of a standard optical fiber will diverge rapidly, a miniature optical system can be coupled to the fiber to provide a focusing or collimating function. Additionally, it is often desirable to analyze a sample location that is not directly in line with the optical axis of the fiber, such as the analysis of the luminal wall of a thin blood vessel. In these situations, a means for substantially altering the direction of the light is used in addition to a means for focusing or collimating the light radiating from the tip of an optical fiber.

There are many miniature optical systems known in the art that can be used for analysis of internal luminal structures. Each optical system can be conceptually divided into a beam focusing means and a beam directing means. Light is passed from an external light source to the internal lumen through one or more optical illumination fibers, which may be single mode or multimode in nature. The illumination fiber is in communication with the miniature optical system, which focuses and directs the beam into the luminal wall.

Several features of existing optical systems are undesirable. For example, in some devices all of the optical elements must be of a diameter similar to the optical fiber in order to minimize the overall system size. This greatly reduces the options available for selecting the focusing element, beam expander, and beam director and therefore limits the range of focal spot sizes and working distances achievable by the design. Additionally, these extremely small elements are fragile, difficult to handle, and prone to break during manufacturing and operation.

In some implementations, an air gap must be provided in order to use total internal reflection (TIR) for beam redirection. This requires a tight seal to be maintained between the fiber and the other element to maintain the air gap. This can be problematic when the device is immersed in water, blood, or stomach acid, or when the device is rotated or translated at high speed in order to form an image.

GRIN focusing elements have refractive index profiles that are rotationally symmetric, making it impossible to correct for cylindrical aberrations induced on the beam. The overall effect of these drawbacks is that certain miniature optical systems are expensive, difficult to manufacture, prone to damage, and do not produce a circular output at the focal plane. A GRIN lens has been viewed as ill-suited to shaping and cutting because imaging artifacts and aberration results when such modifications have been attempted. Polishing a spherical lens to form a beam directing surface is likewise undesirable because unwanted optical aberration results.

Many methods exist to manufacture miniature optical systems suitable for attachment to an optical fiber. Unfortunately, they have numerous inherent limitations, including excessive manufacturing cost, excessive size, or insufficient freedom to select the focal spot size and focal distance. In addition to the drawbacks listed above, conventional lensed surfaces can only provide small radii of curvature and are largely limited to spherical geometries. Additionally, the beam cannot expand to a size significantly larger than the single mode fiber diameter (often 125 μm) at any point in the optical system. These limitations result in a lens system with a limited working distance and significant spherical aberrations.

As described above, there are significant limitations to currently known miniature optical systems used for conducting optical analysis or imaging. Accordingly, a need exists for optical elements that overcome the limitation of existing optical devices. Further, there exists a need in the art for optical probes capable of being used in diagnostic medical devices such as guidewires, torque wires, catheters, endoscopes, bronchoscopes, needles, and trocars. The present disclosure addresses these needs and others.

### SUMMARY

In part, the disclosure relates to a lens assembly that includes an optical fiber and a shaped GRIN lens. The lens assembly can be used to direct light for sensing and imaging and can be incorporated in various devices such as catheters, endoscopes, bronchoscopes, ophthalmic devices and other imaging and sensing systems. In one embodiment, the lens assembly is a component of an intravascular data collection probe such as an optical coherence tomography probe. The lens assembly can include an optical fiber having a first diameter, the optical fiber having a first endface and a second endface and a gradient index lens comprising a rod having a length L.

In one embodiment, the rod can include a substantial planar end and a polished end. The rod can include a longitudinal axis and a second diameter. The second diameter is greater than the first diameter in one embodiment. The substantially planar end is optically coupled such as by a fusion joint or other joint to the second endface. The refractive index changes along the length L of the rod. Further, the polished end includes a beam focusing surface suitable for directing light at an angle relative to the longitudinal axis.

In part, the disclosure relates to a data collection probe and/or a lens suitable for use therewith. In one embodiment, the probe includes a probe tip that includes a GRIN lens comprising a rod having a proximal end and a distal end, the rod having a diameter D_{g} and a length L_{g}; and an optical fiber having a first end and a second end, the optical fiber having a diameter D_{f} and a length L_{f}; wherein the proximal end of the rod is optically and fixedly coupled to the second end of the optical fiber, and wherein the diameter D_{g} of the rod is greater than the diameter D_{f} of the optical fiber.

In one embodiment, the distal end of the rod of the GRIN lens has an angled polished surface such that the distal end of the rod can direct light, the distal end of the rod being grinded to form the angled surface. In one embodiment, the angle of the distal end of the rod is less than about 45 degrees. In one embodiment, the rod of the GRIN lens has a numerical aperture that ranges from about 0.13 to about 0.15. In one embodiment, the diameter D_{g} ranges from about 125 μm to about 250 μm.

In one embodiment, the distal end of the rod of the GRIN lens comprises a beam forming surface oriented at an angle relative to a longitudinal axis of the GRIN lens. In one embodiment, the proximal end of the rod is fixedly coupled to the second end of the optical fiber using an optical adhesive. In one embodiment, the optical adhesive is one of an ultraviolet adhesive, an epoxy, an optical potting material or optically transparent glue. In one embodiment, a beam spot radius focused by the GRIN lens ranges from about 5 μm to about 20 μm

In one embodiment, the first end of the optical fiber is coupled to an OCT system, and wherein light from the OCT system is transmitted along the optical fiber to the rod through an interface between the second end of the optical fiber and the proximal end of the rod. In one embodiment, the first end of the optical fiber is coupled to an OCT system, and wherein light from the OCT system is transmitted along the optical fiber to the rod through an interface between the second end of the optical fiber and the proximal end of the rod.

In part, the disclosure relates to a lens assembly. The lens assembly includes a torque wire having a first end and a second end and defining a bore; an optical fiber having a first diameter secured in the bore, the optical fiber having a first endface and a second endface; and a gradient index lens comprising a rod having a length L_{g}, the rod comprising a substantial planar end and a polished end, the rod having a longitudinal axis and a second diameter, the second diameter greater than the first diameter, the substantially planar end optically coupled to the second endface, wherein refractive index changes along the length L_{g}, wherein the polished end comprises a beam directing surface.

In one embodiment, the beam focusing surface is configured to direct light received from the optical fiber at an angle relative to the longitudinal axis that ranges from about 45° to about 35°. In one embodiment, the beam focusing surface is a non-planar surface configured to reflect light received from the optical fiber through the side of the rod and focus that light at a location outside of the rod. In one embodiment, the length L_{g} ranges from about 1.0 mm to about 2.0 mm. In one embodiment, the second diameter ranges from about 125 μm to about 250 μm. In one embodiment, a reflective coating is disposed on the beam directing surface. In one embodiment, the lens assembly may further include a torque wire defining a bore, wherein at least a portion of the optical fiber and the rod are disposed in the bore.

In part, the disclosure relates to a method of collecting interferometric data from a sample. The method includes transmitting light along an optical fiber such that the light crosses an interface between an optical fiber and a unitary rod comprising a spatially varying index of refraction gradient; focusing light received by the unitary rod with a beam forming surface of the unitary rod on the sample; forming a spot on the sample that ranges from about 5 μm to about 15 μm at a focal length that ranges from about 0.8 mm to about 2.0 mm and detecting light reflected back from the sample through the optical fiber. In one embodiment, the diameter of the unitary rod is greater than about 125 μm and less than about 250 μm.

In part, the disclosure relates to an optical data collection apparatus that includes a rotatable torque coil defining a bore to transmit rotation; an optical fiber optically connectable to a swept laser source at one end and to an optical assembly at the other end, wherein a portion of the optical fiber is disposed in the bore, the optical fiber having a first diameter; an optical assembly includes a unitary polished GRIN lens having a second diameter equal or larger than the first diameter, the optical assembly includes a beam forming or beam directing surface; and a sheath having an optical window positioned to receive light from and send light to the beam forming or directing surface. The sheath may include air, water, glycerol, or contrast solution to improve mechanical and optical performance. The sheath is designed to follow along a guidewire for intravascular positioning. In one embodiment, the optical assembly is an optical probe tip of an optical coherence tomography data collection probe.

The apparatus can include a catheter and a distal portion of the catheter includes the optical assembly with light emitting to side for side viewing from the beam forming or directing surface of the unitary polished GRIN lens. In one embodiment, the optical tip is optical adhesive potted with an end polished GRIN lens and torque coil, the optical adhesive may be UV adhesive, epoxy or any other optical transparent glue.

In one embodiment, the torque coil rotates the potted optical tip for 360° side imaging. In one embodiment, the GRIN lens is formed by fusing a single mode fiber with a shaped GRIN fiber of a selected length. In one embodiment, the side viewing GRIN lens is formed by polishing the end of the GRIN lens into an angle. In one embodiment, the polished angle is less than 45° in order to achieve total-internal-reflection at the polished surface. In one embodiment, the polished GRIN lens is disposed within a heat shrinkable tube to form a total internal reflection surface or coated with high reflectivity metal when the polished surface is potted within an optical glue to form the optical tip.

In one embodiment, the period of the self-focusing GRIN fiber is expressed as Λ=2πn₀a/NA_{grin}. A lower NA_{grin} results in a longer period, where a is the core radius of the GRIN fiber. In one embodiment, the numerical aperture of GRIN fiber equals or exceeds the numerical aperture of the single mode fiber NA_{grin}≥NA_{smf}. In one embodiment, a maximum or relative extrema radius of light rays is expressed as

$${r}_{max} = \frac{{NA}_{smf}}{{n}_{0} \sqrt{2 Δ}} a .$$

In one embodiment, the maximum radius (rₘₐₓ) that light rays can reach is given by rₘₐₓ=a·NA_{smf}/NA_{grin}. In one embodiment, the light rays within the GRIN fiber are restricted within the core of the GRIN fiber when NA_{grin}≥NA_{smf}.

In one embodiment, the GRIN fiber is designed to reduce the amount of light rays emitted from the side of GRIN fiber when a light beam is incident from a single mode fiber. In one embodiment, the focal length of GRIN lens is determined by specifying such as by cutting or molding the GRIN fiber to a certain length. In one embodiment, polishing of GRIN lens reduces the effective length of GRIN lenses and increases the focal length of GRIN lenses. In one embodiment, the length of the GRIN fiber segment is increased by an amount Δd to decrease the focal length.

In one embodiment, the focal length of the polished GRIN lens sub-assembly is approximately related to the average length of the polished GRIN fiber as one side is longer than another side. In one embodiment, a maximum focal length of the shaped GRIN lens segment occurs when the GRIN fiber length satisfies

$$cos ( 2 {gL}_{m} ) = - \frac{{\tilde{w}}^{4} - {{w}_{0}}^{4}}{{\tilde{w}}^{4} + {{w}_{0}}^{4}} or {L}_{m} = [ 2 π - a cos ( \frac{{\tilde{w}}^{4} - {{w}_{0}}^{4}}{{\tilde{w}}^{4} + {{w}_{0}}^{4}} ) ] / ( 2 g ) ,$$

where, g=NA_{grin}/(n₀a), n₀ is the index at the center of GRIN fiber, a is the radius of GRIN fiber, \tilde{w}=\sqrt{λ/(πgn₀)}, w₀ is the radius of mode filed that is incident to the GRIN fiber.

In one embodiment, a maximum focal length is given by

$${f}_{m} = \frac{n}{2 {gn}_{0}} ( \frac{{\tilde{w}}^{2}}{{{w}_{0}}^{2}} - \frac{{{w}_{0}}^{2}}{{\tilde{w}}^{2}} ) .$$

In one embodiment, the radius of the focal spot at the longest focal length is expressed as wₘ=\sqrt{\tilde{w}⁴+w₀⁴}/(\sqrt{2}w₀).

In one embodiment, to reduce the size of the focal spot, the GRIN fiber length is selected to be in the range of

$$L \geq [ 2 π - a cos ( \frac{{\tilde{w}}^{4} - {{w}_{0}}^{4}}{{\tilde{w}}^{4} + {{w}_{0}}^{4}} ) ] / ( 2 g ) ,$$

the focal length is in the range of

$${f}_{m} \leq \frac{n}{2 {gn}_{0}} ( \frac{{\tilde{w}}^{2}}{{{w}_{0}}^{2}} - \frac{{{w}_{0}}^{2}}{{\tilde{w}}^{2}} ) ,$$

and w≤\sqrt{\tilde{w}⁴+w₀⁴}/(\sqrt{2}w₀), respectively. In one embodiment, for \tilde{w}=\sqrt{λ/(πgn₀)}, 2w₀=9.2 μm, Eq.(13) yields Lₘ=1.31 mm, the maximum focal length is obtained from Eq. (14) as fₘ=2.80 mm in air. The radius of beam spot at this maximum focal length is obtained from Eq. (15) as 2w=69 μm. In one embodiment, wherein NA_{grin}=NA_{smf}, the diameter of the GRIN lens fiber segments is increased relative to a SMF to increase the values of fₘ, Lₘ and wₘ.

In one embodiment, the GRIN lens-based lens assemblies are used to increase a resolution of an optical coherence tomography probe such that the resolution ranges from about 0.8 mm to about 2.0 mm.

### BRIEF DESCRIPTION OF THE DRAWINGS

The figures are not necessarily to scale, emphasis instead generally being placed upon illustrative principles. The figures are to be considered illustrative in all aspects and are not intended to limit the invention, the scope of which is defined only by the claims.

FIG. 1 is a schematic diagram of one embodiment of an intravascular data collection system suitable for use with the optical assemblies described herein according to an illustrative embodiment of the disclosure;

FIG. 2A is a side view of one embodiment of a lens assembly for collecting data that includes a GRIN lens fiber segment coupled to a single mode optical fiber according to an illustrative embodiment of the disclosure;

FIG. 2B is a side view of another embodiment of a lens assembly for collecting data that includes a GRIN fiber segment with an angled beam forming surface coupled to a single mode optical fiber according to an illustrative embodiment of the disclosure;

FIG. 2C is a side view of another embodiment of a lens assembly that includes a GRIN fiber segment with a curved beam forming surface coupled to a single mode optical fiber according to an illustrative embodiment of the disclosure;

FIG. 3A is a cross-sectional view of a unitary gradient index rod of a lens assembly according to an illustrative embodiment of the disclosure;

FIG. 3B is a cross-sectional view of a single mode optical fiber suitable for use with a GRIN lens segment in a lens assembly according to an illustrative embodiment of the disclosure;

FIG. 4 is a cross-sectional view of a unitary gradient index rod and an optical fiber of a lens assembly showing the relative diameters of the rod and the optical fiber according to an illustrative embodiment of the disclosure;

FIGS. 5A and 5B are side views of embodiments of a GRIN lens fiber segment and an optical fiber positioned inside a torque wire with a surround flexible sheath according to an illustrative embodiment of the disclosure;

FIG. 6 is a schematic diagram of a ray tracing simulation showing periodic self-focusing a light as light rays propagate along a GRIN fiber according to an illustrative embodiment of the disclosure;

FIG. 7 is a schematic diagram of a ray tracing showing outer light rays as the light rays reach a boundary of a GRIN fiber according to an illustrative embodiment of the disclosure;

FIG. 8 is a schematic diagram of a ray tracing of various features of light as produced using one embodiment of a GRIN lens according to an illustrative embodiment of the disclosure;

FIG. 9 is a plot of focal length and diameter of a focal spot versus a length of one embodiment of a GRIN lens;

FIG. 10A is a plot of a length of a GRIN lens and focal length versus diameter of a GRIN lens according to an illustrative embodiment of the disclosure;

FIG. 10B is a plot of diameter of a focal spot versus diameter of a GRIN lens according to an illustrative embodiment of the disclosure;

FIGS. 11A and 11B show a simulation of one embodiment of a GRIN lens and a diameter of the produced focal spot according to an illustrative embodiment of the disclosure;

FIG. 12 is one embodiment of GRIN lens with a polished angled end according to an illustrative embodiment of the disclosure;

FIG. 13A is a phantom test image showing an image resolution for a multi-piece GRIN lens according to an illustrative embodiment of the disclosure;

FIG. 13B is a phantom test image showing an image resolution for one embodiment of a unitary polished GRIN lens according to an illustrative embodiment of the disclosure;

FIG. 14A is an artery test image showing an image resolution of the inside of a vessel for a multi-piece GRIN lens according to an illustrative embodiment of the disclosure;

FIG. 14B is an artery test image showing an image resolution of the inside of a vessel for one embodiment of a unitary polished GRIN lens according to an illustrative embodiment of the disclosure;

FIG. 15A is an artery test image with stent struts showing an image resolution of the inside of a vessel for a multi-piece GRIN lens according to an illustrative embodiment of the disclosure; and

FIG. 15B is an artery test image with stent struts showing an image resolution of the inside of a vessel for one embodiment of a one-piece polished GRIN lens according to an illustrative embodiment of the disclosure.

### DETAILED DESCRIPTION

In part, various systems and methods of collecting data using an optical fiber-based probe are disclosed. These probes include one or more optical elements to direct light for sensing or imaging applications such as sensing or imaging directed to tissue, fabricated materials and structures, and other physical objects. The probes can be used as part of an intravascular data collection probe such as an optical coherence tomography (OCT) probe in one embodiment. The arrangement of and the type of optical elements use to receive and direct light is one aspect of the disclosure.

The use of a GRIN lens that includes a rod having a substantially planar surface and beam forming surface as a unitary or one-piece optical element in lieu of two or more spliced optical elements offers optical and manufacturing advantages. In one embodiment, a GRIN lens formed from a shaped rod is coupled to an optical fiber such that the rod and optical fiber have differently sized diameters. The use of a GRIN lens optically coupled to an optical fiber as part of a probe offers advantages over existing probe designs as outlined herein.

Some existing data collection probe designs that employ light directed to and received from a side of the probe include three components. These three components are joined together to form a lens assembly such as a GRIN lens assembly. In turn, this GRIN lens assembly is then placed in optical communication with an optical fiber such as a single mode fiber (SMF) that sends light to and receives scattered light collected by the probe such as by an optical fiber. The three components of a conventional lens assembly include (1) a coreless fiber also referred to a “no core” fiber (NCF) which is used as a beam expander, (2) a graded index (GRIN) fiber which is used as a lens to focus light and (3) an angled beam director or prism such as an angle polished NCF. The lengths of these three components need to be precisely controlled. This creates various manufacturing challenges.

Given the required precision needed to fuse three components of the size scale of optical fibers, the manufacturing costs are significant. Further, under certain circumstances the fusion joints between the three fiber segments degrade the optical performance as a result of light having to transition through the different joints. In part, the disclosure relates to a lens assembly that is formed from a unitary GRIN lens. A unitary approach avoids splicing separate segments and components together.

In one embodiment, a single rod having continuous refractive index changes along its length is processed, such as by polishing, molding or cleaving, to create a beam forming surface rather than fuse or join three separate components as described above. The unitary GRIN lens is typically sized such that its diameter ranges from 125 um to 250 um and thus is greater than or equal to the diameter of the single mode optical fiber to which it is optically and/or fixedly coupled. Thus, a polished unitary GRIN lens can be used as part of a sensing, imaging or other data collection probe to significantly improve the optical performance and reduce the manufacturing cost associated with multi-component lens assemblies.

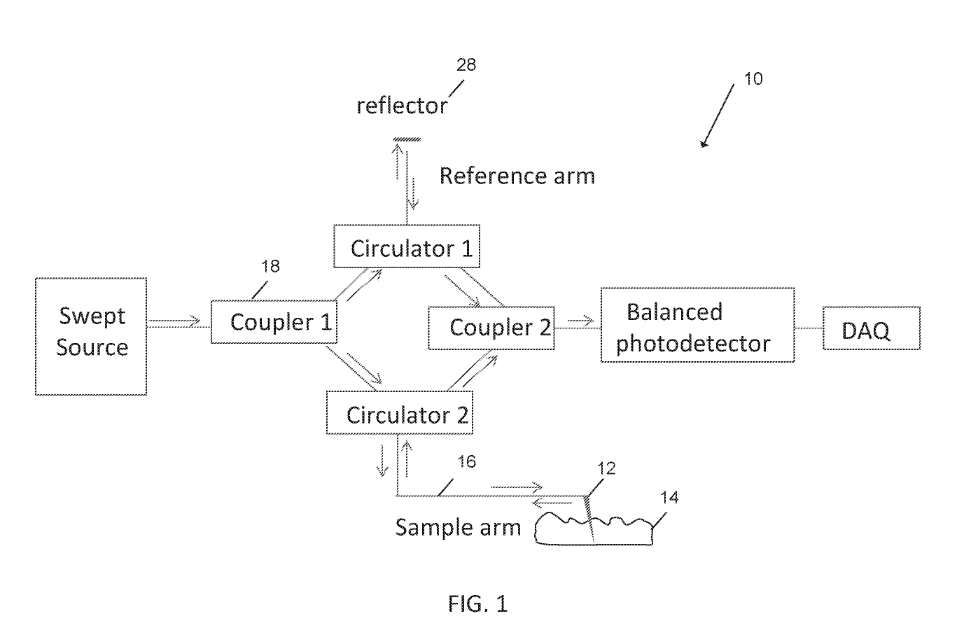

In part, the disclosure relates to probes that include an optical assembly having suitable light directing and focusing optical components such as a GRIN lens such that data can be collected by imaging to the side of a longitudinal axis of an optical fiber disposed in the probe. These probes can be used for various sensing and light directing applications. In one embodiment, the probes are suitable for use with an interferometric imaging system such as the exemplary system described herein and depicted in FIG. 1.

In FIG. 1, an image data collection system 10 is shown that includes a data collection probe having an optical fiber that can be connected to the system 10 via various mechanisms such that it forms part of the sample arm and has a probe tip 12 suitable for directing and receiving light from a sample 14. The probe tip 12 typically includes a GRIN-lens based assembly as described herein. The system can include an interferometer having a reference arm and a sample arm. The optical fiber 16 can be part of the sample arm of the interferometer, and a reflector 28 such as a movable mirror on a track is part of the interferometer and specifies one terminus of the reference arm. A first circulator and a second circulator as shown can be optically coupled to a first optical coupler and a second optical coupler as shown. The first optical coupler can be in communication with a swept source of electromagnetic radiation.

A balanced photodetector is in optical communication with the second coupler. A data acquisition device or DAQ is communication with the balanced photodetector. The reflector 28 is in optical communication with a first circulator. The sample arm portion connected to the probe and probe tip 12 is in optical communication with a second circulator. The two circulators are in optical communication with the first and second couplers. This is but one system embodiment 10 and various data collection systems can be used with the lens assembly and probes described herein.

In one embodiment, a swept source, is configured to produce light that passes by way of an optical path into the first optical coupler. Light entering the first coupler 18 is the split along an optical fiber path to the first circulator and a path to the second circulator. One path from the first circulator terminates at the reflector 28, while the sample arm portion from the second circulator enters an optical fiber and transmitted to the probe tip. The probe allows light to be directed into a sample such as vascular tissue, for example, to a wall of a vessel within the tissue.

The balanced photodetector can have a variety of configurations, for example, such as a photodiode. The output signal from the detector is processed by a processor or other components of an OCT system such as a DAQ connected thereto. In one embodiment, the OCT system is a workstation or server configured to run one or more software modules and process frames or scan lines of image data corresponding to cross-sections of the vessel showing features of the vessel wall.

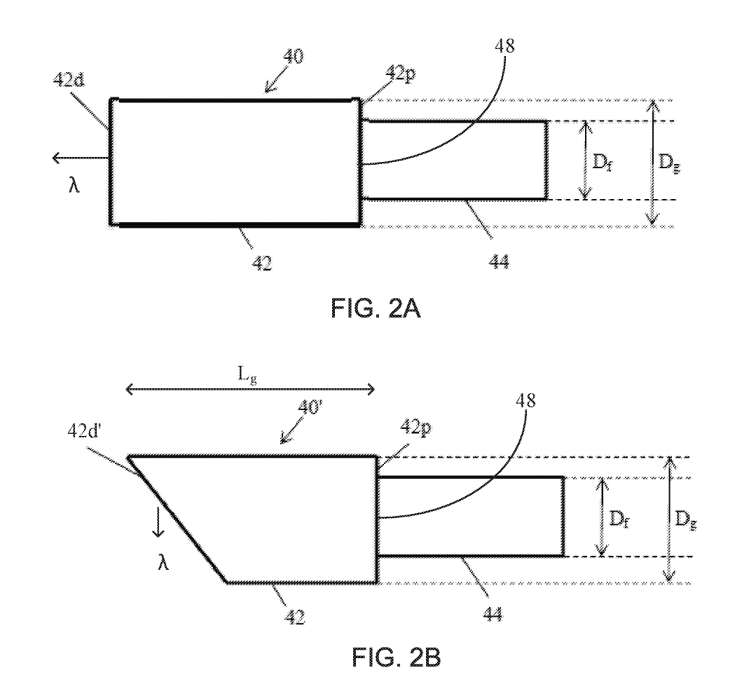

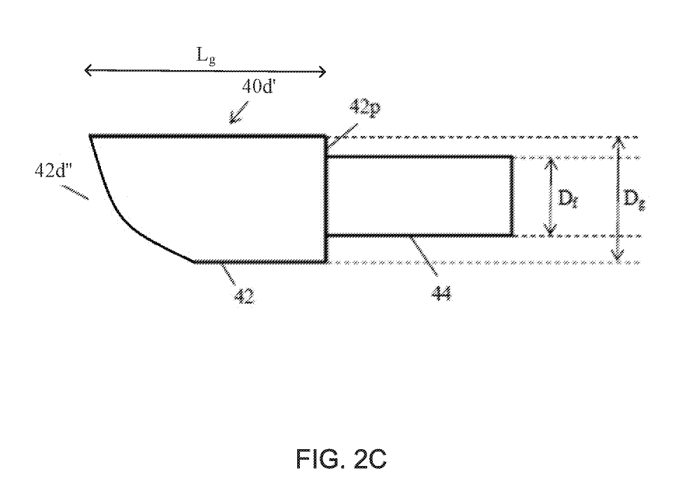

FIGS. 2A-2C illustrate exemplary embodiments of a probe tip having a beam directing surface (42d, 42d′, and 42d″) of a data collection probe that generally includes an optical fiber having a first end configured to couple to a data collection system such as an OCT system and a second end configured to be coupled to a GRIN lens assembly of the disclosure. In one embodiment, shown in FIG. 2A, a GRIN lens 40 includes a rod 42 having a proximal end 42p and a distal end 42d. In one embodiment, the rod is a NCF such as a coreless or unitary optical fiber segment that is manufactured to have a spatially varying gradient index. The proximal end 42p of the rod 42 is fixedly coupled to a second end 48 of an optical fiber 44. The distal end 42d is configured to direct light that has propagated along the optical fiber 44 and the rod 42 of the GRIN lens 40 towards a sample or object of interest. Light is typically directed to the side of the probe from the probe tip using a curved or angled surface of GRIN lens 40 to direct the light.

The rod 42 can have a variety of configurations, and, for example, can be in the form of an elongate rod having a diameter D_{g} and a length L_{g}. The proximal end 42p of the rod 42 is configured such that it is suitable to be coupled to the optical fiber 44. In one embodiment, the proximal end 42p of the rod 42 of the GRIN lens 40 expands light to a desired diameter.

The distal end 42d of the rod 42 is configured to allow light to pass to a vessel in the tissue. The distal end 42d of the rod 42 can have a variety of angles relative to the longitudinal axis of the rod. For example, in one embodiment shown in FIG. 2A, the distal end 42d of the rod 42 is configured such that the surface of the distal end 42d of the rod 42 is substantially perpendicular to the length of the rod 42 and thus has a 90 degree angle with respect to the longitudinal axis of the rod 42. In FIG. 2A, light λ is shown exiting a substantially planar beam forming or beam directing surface 42d in a direction parallel to the longitudinal axis of the fiber. The length of the GRIN lens fiber segment is indicated as Lg and can range from about 1.0 mm to about 2.0 mm.

In another embodiment shown in FIG. 2B, a distal end 42d′ of a rod 42′ is configured such that the surface of the distal end 42d′ is angled with respect to the length of the rod 42′. The angle of the distal end 42d′ of the rod 42′ can be achieved in a variety of ways, including grinding down the distal end 42d′ of the rod 42′ to create a desired angle. The distal end 42d of the rod 42 is configured to focus light to form a spot having a beam spot radius that ranges from about 5 um to about 20 um.

Thus, the angle of the distal end 42d of the rod 42 can be chosen based on the desired beam spot radius. For example, the angle of the distal end 42d of the rod 42 can be less than 45 degrees to allow for total internal reflection at the distal end 42d of the rod 42. In FIG. 2B, light λ is shown exiting an angled beam forming or beam directing surface 42d′ in a direction perpendicular to the longitudinal axis of the fiber. In another embodiment shown in FIG. 2C, rod 42″ has a distal end 42d″ that has been polished or otherwise shaped to include a curved beam forming surface.

In an illustrated embodiment, the optical fiber 44 has a diameter D_{f} and a length L_{f} such that the length allows a second end of the optical fiber to be coupled to the an OCT system, while a first end of the optical fiber is coupled to the rod 44 with having a length sized to allow the optical fiber 44 is be inserted into a vascular tissue for the collection of data therein.

The rod or cylindrical solid that is doped to have the gradient index desired can be polished or otherwise shaped to form a unitary GRIN lens. The unitary GRIN lens and the optical fiber can be coupled together in a variety of ways as part of various probe designs. For example, in one embodiment, the proximal end of the rod is fixedly coupled to the second end of the optical fiber using a variety of techniques, including the use of an optical adhesive, such as an optically transparent glue, optical potting material, splicing or butt coupling.

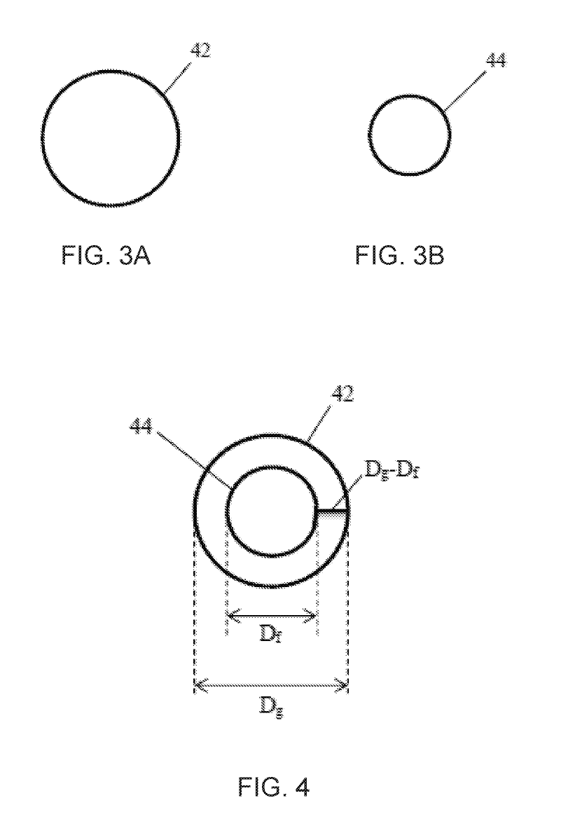

FIGS. 2A-2B also illustrated the relative diameters of the optical fibers and the GRIN lens. For example, as shown in FIG. 2A (and also in FIG. 2B), the diameter D_{g} of the rod 42, shown in cross section in FIG. 3A, is greater than the diameter D_{f} of the optical fiber 44, shown in cross section in FIG. 3B. The relative difference between the two diameters is shown in FIG. 4.

The ratio of the GRIN lens diameter D_{g} to the optical fiber diameter D_{f} can range from about 1 to about 2 in one embodiment. The absolute value of the difference of the diameter D_{g} and the optical fiber diameter D_{f} can range from 0 about to about 125 um in one embodiment. The diameter of the GRIN lens fiber segment is indicated as Dg and can range from about 125 to about 250 um.

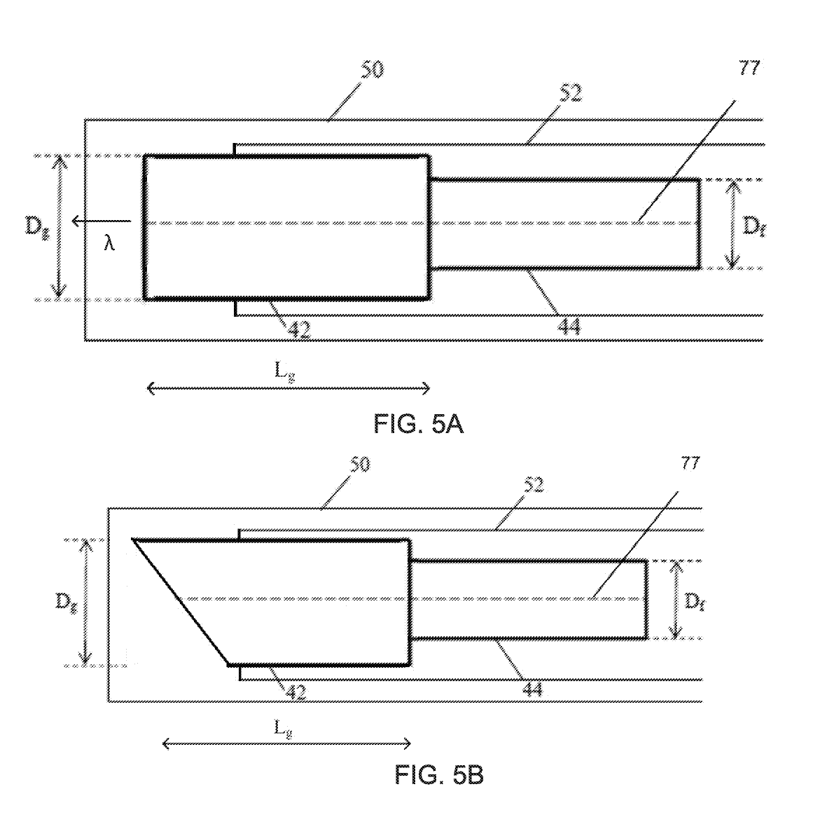

FIGS. 5A and 5B illustrate an embodiment of a GRIN lens and an optical fiber inserted in a sheath with a torque wire for inserting the device into vascular tissue. In FIG. 5A the beam forming or directing surface 42d is substantially planar and in FIG. 5B it is angled as shown. The second end of the optical fiber and the GRIN lens are disposed within one or more sheaths such as a sheath 50 shown in FIGS. 5A and 5B. The longitudinal axis 77 is also shown in each of the figures.

The optical fiber 44 and rod 42 of the GRIN lens can also include a torque wire 52 defining a bore to receive optical fiber 44. For example, in one embodiment, the rod 42 of the GRIN lens and the optical fiber 44 can be moved through a vessel in a tissue such that the optical fiber 44 and the rod 42 positioned within the sheath 50 can rotate and the beam of light sent to the vessel from the distal end of the rod 42 traces a spiral as it moves along the section of the vessel being imaged. As a result, different images are obtained with regard to different sections of the vessel. The lens assemblies and shaped GRIN lens described herein can be used in different data collections systems and different applications

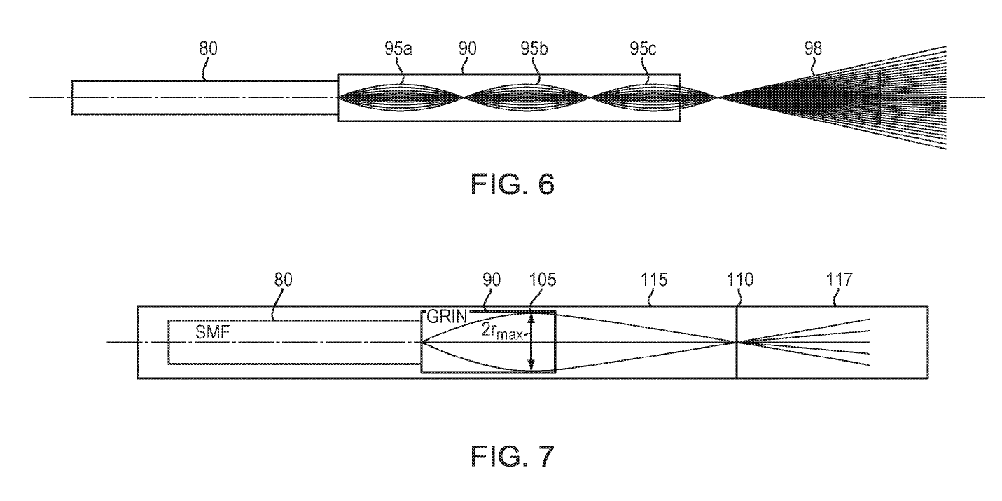

In FIG. 6, a single mode optical fiber 80 is shown fixedly and optically coupled to a GRIN lens fiber segment or rod 90 that includes a gradient index. The light rays propagate along the graded index fiber 90 as shown in FIG. 6 when the refractive index at the cross section of the fiber is a parabolic profile. The simulation shown in FIG. 6 shows a periodic self-focusing when light rays propagate along a GRIN fiber (NA_{smf}=0.11, NA_{grin}=0.22). As can be seen in the schematic diagram of FIG. 6, the light entering from single mode fiber 80 expands and focuses to a point within three distinct regions 95a, 95b, and 95c.

The parabolic refractive index profile of a GRIN fiber is usually expressed as

$$n ( r ) = \{ \begin{matrix}{n}_{0} \sqrt{1 - 2 {( r / a )}^{2} Δ} & ( r \leq a ) \\ {n}_{2} & ( r > a )\end{matrix} \qquad ( 1 )$$

Where a and r are the radiuses of fiber core and light rays, respectively, (0≤r≤a); n₀ and n₂ are the refractive indexes of fiber center and cladding, respectively.

The parameter of Δ is expressed as

Δ=NA_{grin}²/(2n₀²) (2)

where NA_{grin}=\sqrt{n₀²−n₂²} is the numerical aperture of the GRIN fiber.

Equation (1) can be expressed in another form as

n(r)=n₀\sqrt{1−g²r²}(r≤a) (3)

where g is the called quadratic index constant, expressed as

$$g = \frac{{NA}_{grin}}{{n}_{0} a} \qquad ( 4 )$$

The period of the self-focusing is given by Λ=2π/g and expressed as

$$Λ = \frac{2 π {n}_{0} a}{{NA}_{grin}} \qquad ( 5 )$$

When a ray is incident into the core of a GRIN fiber from a single mode fiber at an angle of θ₀, this ray will reach its maximum radius rₘₐₓ and then changes its direction as shown by the upper and lower rₘₐₓ values that give 2 rₘₐₓ in FIG. 7. After reaching a rₘₐₓ value in GRIN lens segment 90 at a distance 105 the rays then decrease in region 115 to a focus at position 110 until they resume their expansion in region 117 as shown in FIG. 7. The ray equations can be expressed as

$$\{ \begin{matrix}{NA}_{smf} = {n}_{0} sin ( {θ}_{0} ) \\ {n}_{0} cos ( {θ}_{0} ) = {n}_{0} \sqrt{1 - 2 {( {r}_{max} / a )}^{2} Δ}\end{matrix} \qquad ( 6 )$$

where NA_{smf} is the numerical aperture of single mode fiber. Simplifying equation (6), the maximum radius that the light rays can reach is obtained as

$${r}_{max} = \frac{{NA}_{smf}}{{n}_{0} \sqrt{2 Δ}} a \qquad ( 7 )$$

Using Eq. (2), Eq. (7) becomes

$${r}_{max} = \frac{{NA}_{smf}}{{NA}_{grin}} a \qquad ( 8 )$$

When NA_{smf}>NA_{grin}, the outer light rays will exit the core of the GRIN fiber, while when NA_{smf}<NA_{grin}, all light rays are restricted within the fiber core (rₘₐₓ<a). However, when NA_{smf}<<NA_{grin}, the light rays do not fully occupy the core of GRIN fiber. As a result, the real optical aperture is reduced and the effective focal length is shortened. In order to increase the optical aperture, a piece of coreless fiber as a beam expander is spliced between the single mode fiber and the GRIN fiber to expand the beam to cover the core of GRIN fiber. When NA_{smf}=NA_{grin} the outer rays just reach the boundary of the GRIN fiber core and cladding, as shown in FIG. 7. As a result, the maximum ray radius is obtained as

rₘₐₓ=a|_{NA}_{_{smf}}_{=NA}_{_{grin}} (9)

The schematic diagram representing simulation of ray tracings for light exiting optical fiber 80 and being received by GRIN lens segment 90 in FIG. 7 shows that the outer light rays just reach the boundary of the GRIN fiber core and cladding when their numerical apertures are matched as NA_{smf}=NA_{grin}.

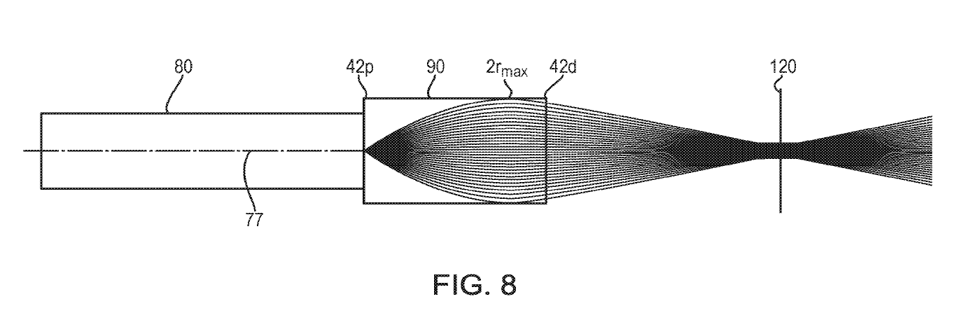

As shown in the schematic diagram of FIG. 8, which is simulation of light traveling along longitudinal axis in a fiber core of fiber 80 before reaching a first surface 42p of GRIN lens 90 and then existing beam forming surface 42d, for a single mode fiber 80 and GRIN fiber 90 with parameters of D_{smf}=9 μm, NA_{smf}=0.11, D_{grin}=180 μm, NA_{grin}=0.115. As shown in the simulation of FIG. 8, the maximum diameter of rays given by Equation (8) is obtained as 2rₘₐₓ=172 μm, slightly smaller than the core diameter. The beams are focused at position 120 as shown. 2rₘₐₓ is depicted in FIG. 8 according to one embodiment of the disclosure.

The simulation shown in FIG. 8 of unitary GRIN lens with parameters of: D_{smf}=9 μm, NA_{smf}=0.11, D_{grin}=180 μm, NA_{grin}=0.115, L_{grin}=2.125 mm, the simulation shows the maxima beam diameter 2rₘₐₓ=172 μm, focal length 3 mm in water, the diameter of focal point at 1/e² is 21 μm.

The focal length is expressed as

$$f = \frac{n ( 1 - \frac{{\tilde{w}}^{4}}{{{w}_{0}}^{4}} ) sin ( gL ) cos ( gL )}{{gn}_{0} [ {sin}^{2} ( gL ) + \frac{{\tilde{w}}^{4}}{{{w}_{0}}^{4}} {cos}^{2} ( gL ) ]} \qquad ( 10 )$$

where, \tilde{w}=\sqrt{λ/(πgn₀)}, λ is light wavelength in vacuum, w₀ is the mode radius of incident light. When a single mode fiber (SMF) such as a SMF28 is spliced with a GRIN fiber, this mode diameter 2w₀=9.2 um, L is the GRIN fiber length. n₀ is the refractive index at GRIN fiber center. The radius of the beam waist at the focal spot is given as

$$w = \frac{λ}{π {gn}_{0} {w}_{0} \sqrt{{sin}^{2} ( gL ) + \frac{{\tilde{w}}^{4}}{{{w}_{0}}^{4}} {cos}^{2} ( gL )}} \qquad ( 11 )$$

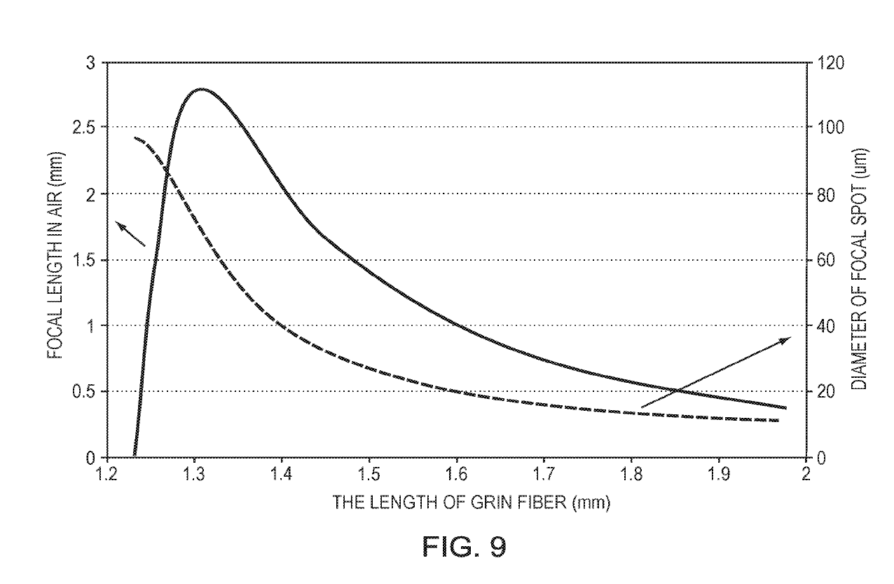

For NA_{grin}=0.14, D_{grin}=150 μm, the focal length and the diameter of the focal spot (2w) with respect to the GRIN fiber length in air is shown in FIG. 9. For L=1.50 mm, the focal length f=2.05 mm, spot diameter is 2w=26.9 μm. There is a longest focal length achievable as fₘ=2.80 mm when Lₘ=1.31 mm, where the diameter of beam waist is 2wₘ=69 μm.

FIG. 9 illustrates the theoretical plots of the focal length and the diameter of focal spot when a SMF such as a SMF28 is spliced with a GRIN fiber. The black solid curve shows the focal length with respect to the GRIN fiber length, and the dashed curve shows the diameter of beam waist at focal spot with respect to the GRIN fiber length; showing when L=1.5 mm, the focal length f=2.05 mm, 2w=26.9 μm. The longest focal length is fₘ=2.80 mm while the diameter of focal spot 2wₘ=69 μm when Lₘ=1.31 mm. (NA_{grin}=0.14, D_{grin}=150 μm).

When the differential of Eq. (10) with respect to the GRIN fiber length is zero, df/dL=0, the longest focal length is achieved and the GRIN fiber length L is given by Eq. (12) as

$$cos ( 2 {gL}_{m} ) = - \frac{{\tilde{w}}^{4} - {{w}_{0}}^{4}}{{\tilde{w}}^{4} + {{w}_{0}}^{4}} \qquad ( 12 )$$

where, g=NA_{grin}/(n₀a). Since f>0, Eq. (10) requires sin(2gLₘ)<0, the corresponding GRIN fiber length is obtained as

$${L}_{m} = [ 2 π - a cos ( \frac{{\tilde{w}}^{4} - {{w}_{0}}^{4}}{{\tilde{w}}^{4} + {{w}_{0}}^{4}} ) ] / ( 2 g ) \qquad ( 13 )$$

The longest focal length of this GRIN lens is expressed as

$${f}_{m} = \frac{n}{2 {gn}_{0}} ( \frac{{\tilde{w}}^{2}}{{{w}_{0}}^{2}} - \frac{{{w}_{0}}^{2}}{{\tilde{w}}^{2}} ) \qquad ( 14 )$$

The corresponding radius of the beam spot at this longest focal length is obtained as

$${w}_{m} = \frac{\sqrt{{\tilde{w}}^{4} + {{w}_{0}}^{4}}}{\sqrt{2} {w}_{0}} \qquad ( 15 )$$

For \tilde{w}=\sqrt{λ/(πgn₀)}, 2w₀=9.2 μm, NA_{grin}=0.14, D_{grin}=150 μm, Eq.(13) yields Lₘ=1.31 mm, the longest focal length is obtained from Eq. (14) as fₘ=2.80 mm, while the radius of beam spot at this focal length given by Eq. (15) as 2wₘ=69 μm. To achieve a small the spot beam size and the longer focal length, as shown in FIG. 9, the GRIN lens' length is selected such that L≥Lₘ, the focal length and spot radius are in the range of f≤fₘ and w≤wₘ, respectively.

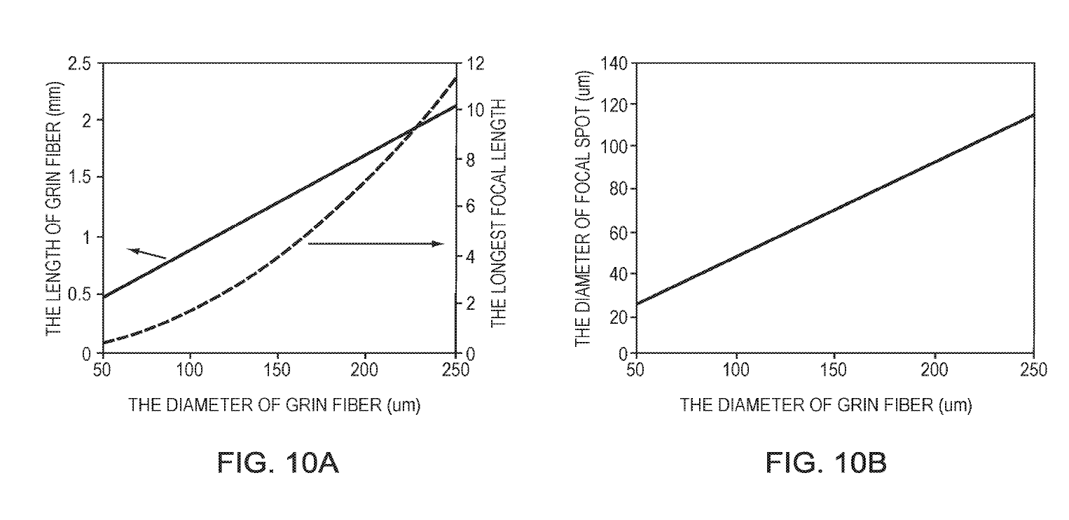

For parameters of NA_{smf}=NA_{grin}=0.14, when the diameter of GRIN fiber varies from 50 um to 250 um, the corresponding Lₘ, fₘ and wₘ are calculated from Eq. (13), (14) and (15) and plotted in FIGS. 10A-B. From a design standpoint, increasing the diameter of the GRIN fiber yields longer fₘ, Lₘ and larger wₘ, respectively. As a result, the size of the diameter can be selected to select fₘ, Lₘ, and wₘ values.

FIGS. 10A-B show the theoretical plot of Eq. (13), (14) and (15) with respect to the diameter of GRIN fiber. FIG. 10A shows the length of GRIN fiber and longest focal length versus the diameter of GRIN fiber, and FIG. 10B shows the diameter of focal spot versus the diameter of GRIN fiber. In one embodiment, the fₘ value of the GRIN lens ranges from about 0.8 mm to about 2.0 mm. In one embodiment, the Lₘ value of the GRIN lens ranges from about 1.9 mm to about 1.5 mm. In one embodiment, the wₘ value of the GRIN lens ranges from about 5 μm to about 20 μm.

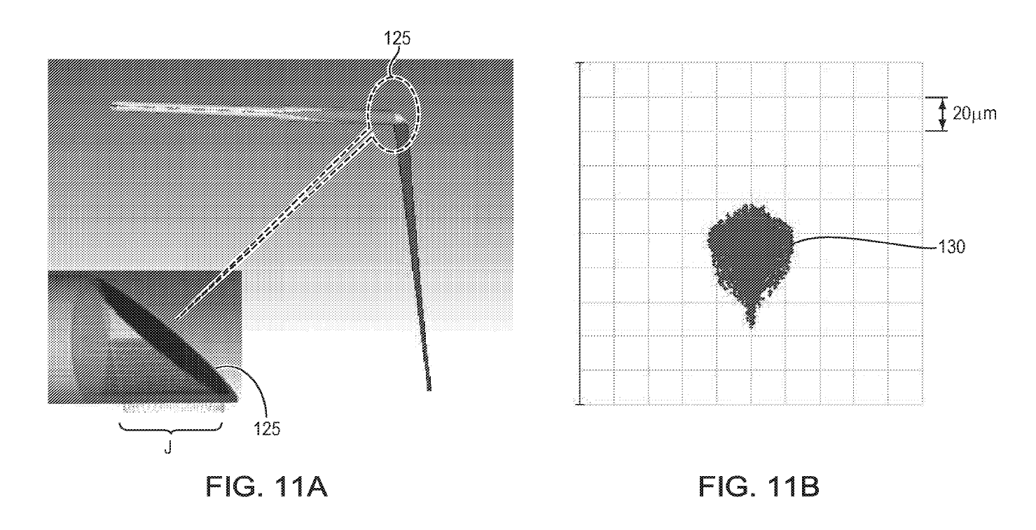

FIGS. 11A-11B show a computer simulation of a unitary GRIN lens assembly having a polished beam forming surface 125 suitable for sending and receiving light from the side of a data collection probe. The parameters of this lens assembly are as follows: D_{smf}=9 μm, NA_{smf}=0.14, D_{grin}=180 μm, NA_{grin}=0.14, L_{grin}=1.5 mm. The simulation shows the RSM diameter of focal spot is 34.0 μm.

To direct light at an angle relative to the longitudinal axis of the GRIN lens fiber segment, the GRIN lens fiber segment can be polished to form an angle or a beam forming surface such as a prism, a curve surface or other optical shaped element, as shown in FIG. 11A. A polished angled beam director 125 or prism formed at the end of the GRIN fiber will result in some optical distortion as light rays travel different lengths due to the angle polishing. A computer simulation shows that the pattern of the focal spot 130 becomes slightly distorted from the circle shape as shown in FIG. 11B. The root-mean-squared (RMS) spot diameter is about 34.0 μm.

When a GRIN lens is spliced with a coreless fiber segment for polishing to form the beam director, the optical simulation shows that the focal spot becomes a circle shape, and the RMS diameter is 30.8 μm. As a result, the RMS diameter becomes about 10% distorted due to polishing beam director surface on GRIN fiber as compared with polishing prism on NCF. However, polishing a cylindrical or rod-shaped GRIN lens avoids a splicing join of the GRIN lens and a NCF beam director such as an optical fiber-based prism or angled reflector. Polishing the GRIN lens also facilitates compensation for optical distortion.

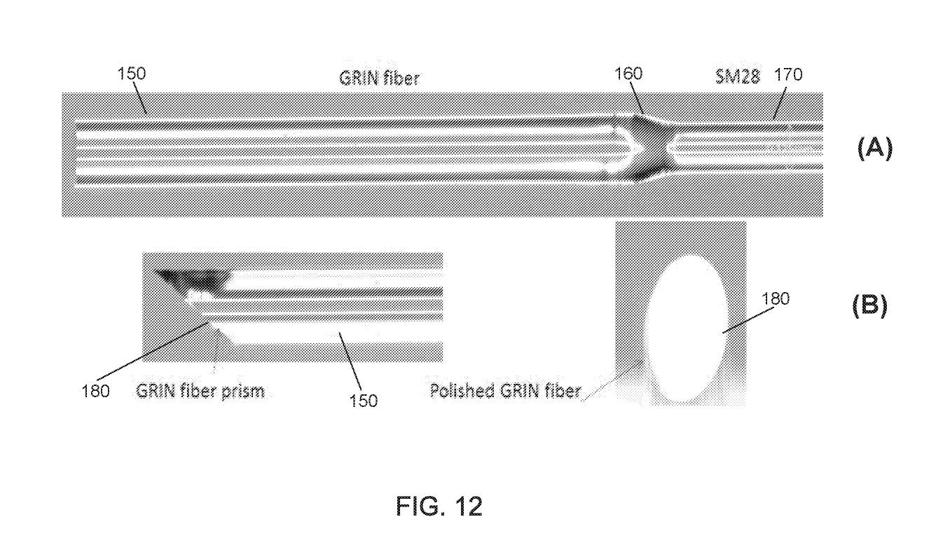

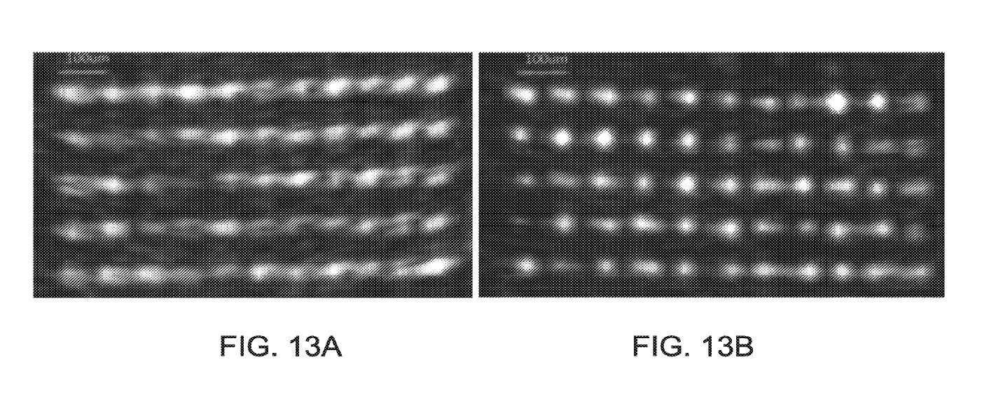

The GRIN lenses were tested and compared with commercial OCT imaging catheters as shown in FIGS. 13A-15B. As a result of the comparison there are indications that the unitary larger GRIN lens OCT catheters have better resolution compared to some existing OCT imaging catheters. As shown in the top portion (A) of FIG. 12, prior to undergoing polishing or shaping, a unitary GRIN lens 150 is shown in FIG. 12 fixedly and optically joined to an optical fiber 170 at join 160. In the bottom portion (B) of FIG. 12, the GRIN lens fiber 150 has been clamped and polished to form an angled beam directing surface 180 into an angle to achieve a flattened polished surface. In one embodiment, a reflective coating can be applied to surface 180. In one embodiment, no coating is necessary because total internal reflection (TIR) provides sufficient reflection with respect to a surrounding air pocket.

The resolution imaging test is shown in FIGS. 13A-B. A phantom was imaged using the GRIN lens embodiments described herein and other OCT imaging proves. The phantom used was pure silica glass with laser etched dots with spacing of about 60 μm to about 70 μm. This phantom is designed to facilitate testing and inspection of the resolution of OCT catheters and the lens assembly of their probe tips. When the OCT probe is set at 1.5 mm away from the phantom, unitary polished GRIN lens can clearly distinguish the etched dots as shown in FIG. 13B. The resolution is significantly improved relative to the commercial OCT probes, as shown in FIG. 13A.

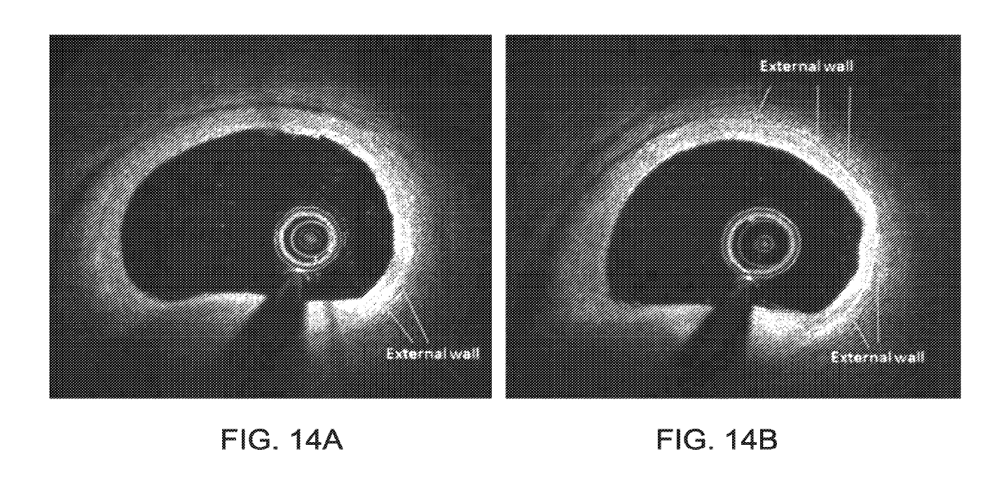

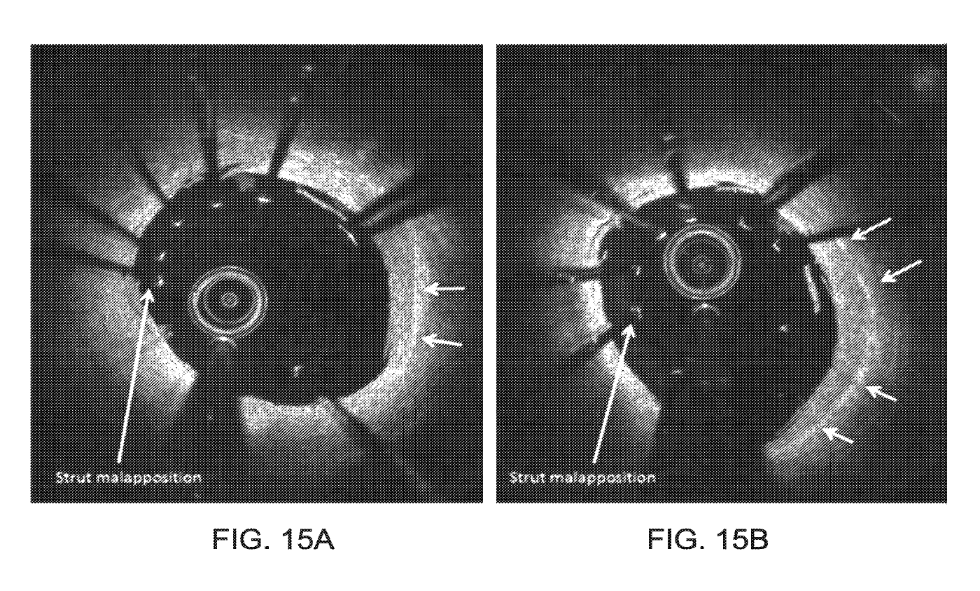

For artery imaging, as shown in FIGS. 14A-15B, unitary GRIN lens OCT catheters also have demonstrable resolution enhancements and imaging depth improvements when compared with existing OCT catheter designs. As shown in FIG. 14B the GRIN len embodiment disclosed herein shows the long structure indicating the artery external surface as marked. In contrast, this structure is shown as short and dim in FIG. 14A which uses a conventional multicomponent lens assembly. FIG. 15B, which uses the GRIN lens of the disclosure, shows a better imaging depth comparing with the conventional OCT imaging probe using a fused three component lens assembly as shown in FIG. 15A. Strut malapposition is shown in both FIGS. 15A and 15B. The arrows on the outer edge also show edges or boundaries associated with the blood vessel. As a result, this disclosure provides various GRIN-lens based optical assemblies that can enhance imaging systems such as intravascular imaging systems.

The aspects, embodiments, features, and examples of the invention are to be considered illustrative in all respects and are not intended to limit the invention, the scope of which is defined only by the claims. Other embodiments, modifications, and usages will be apparent to those skilled in the art without departing from the spirit and scope of the claimed invention.

The use of headings and sections in the application is not meant to limit the invention; each section can apply to any aspect, embodiment, or feature of the invention.

Throughout the application, where compositions are described as having, including, or comprising specific components, or where processes are described as having, including or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components and can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes,” “including,” “have,” “has,” or “having” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions may be conducted simultaneously.

Where a range or list of values is provided, each intervening value between the upper and lower limits of that range or list of values is individually contemplated and is encompassed within the invention as if each value were specifically enumerated herein. In addition, smaller ranges between and including the upper and lower limits of a given range are contemplated and encompassed within the invention. The listing of exemplary values or ranges is not a disclaimer of other values or ranges between and including the upper and lower limits of a given range. As used herein, the term “about” refers to a ±10% variation from the nominal value. As used herein, the term “substantially” refers to a ±10% variation from the nominal value.

It is to be understood that the figures and descriptions of the invention have been simplified to illustrate elements that are relevant for a clear understanding of the invention, while eliminating, for purposes of clarity, other elements. Those of ordinary skill in the art will recognize, however, that these and other elements may be desirable. However, because such elements are well known in the art, and because they do not facilitate a better understanding of the invention, a discussion of such elements is not provided herein. It should be appreciated that the figures are presented for illustrative purposes and not as construction drawings. Omitted details and modifications or alternative embodiments are within the purview of persons of ordinary skill in the art.

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

What is claimed is:

1. A data collection apparatus comprising:
a probe tip comprising:
a GRIN lens comprising a rod having a proximal end and a distal end, the rod having a diameter D_{g} and a length L_{g}; and
an optical fiber having a first end and a second end, the optical fiber having a diameter D_{f} and a length L_{f};
wherein the proximal end of the rod is optically and fixedly coupled to the second end of the optical fiber, and
wherein the diameter D_{g} of the rod is greater than the diameter D_{f} of the optical fiber.

2. The apparatus of claim 1, wherein the distal end of the rod of the GRIN lens has an angled polished surface such that the distal end of the rod is configured to direct light, the distal end of the rod being grinded to form the angled surface.

3. The apparatus of claim 2, wherein the angle of the distal end of the rod is less than about 45 degrees.

4. The apparatus of claim 1, wherein the rod of the GRIN lens has a numerical aperture that ranges from about 0.13 to about 0.15.

5. The apparatus of claim 1, wherein diameter D_{g} ranges from about 125 μm to about 250 μm.

6. The apparatus of claim 1, wherein the distal end of the rod of the GRIN lens comprises a beam forming surface oriented at an angle relative to a longitudinal axis of the GRIN lens.

7. The apparatus of claim 1, wherein the proximal end of the rod is fixedly coupled to the second end of the optical fiber using an optical adhesive.

8. The apparatus of claim 7, wherein the optical adhesive is one of an ultraviolet adhesive, an epoxy, an optical potting material or optically transparent glue.

9. The apparatus of claim 1, wherein a beam spot radius focused by the GRIN lens ranges from about 5 μm to about 20 μm.

10. The apparatus of claim 1, wherein the first end of the optical fiber is coupled to an OCT system, and wherein light from the OCT system is transmitted along the optical fiber to the rod through an interface between the second end of the optical fiber and the proximal end of the rod.

11. The apparatus of claim 1, wherein the first end of the optical fiber is coupled to an OCT system, and wherein light from the OCT system is transmitted along the optical fiber to the rod through an interface between the second end of the optical fiber and the proximal end of the rod.

12. A lens assembly comprising:
a torque wire having a first end and a second end and defining a bore;
an optical fiber having a first diameter secured in the bore, the optical fiber having a first endface and a second endface; and
a gradient index lens comprising a rod having a length L_{g}, the rod comprising a substantial planar end and a polished end, the rod having a longitudinal axis and a second diameter, the second diameter greater than the first diameter,
the substantially planar end optically coupled to the second endface, wherein refractive index changes along the length L_{g},
wherein the polished end comprises a beam directing surface.

13. The lens assembly of claim 12 wherein the beam directing surface is configured to direct light received from the optical fiber at an angle relative to the longitudinal axis that ranges from about 45° to about 35°.

14. The lens assembly of claim 12 wherein the beam focusing surface is a non-planar surface configured to reflect light received from the optical fiber through a side of the rod and focus that light at a location outside of the rod.

15. The lens assembly of claim 12 wherein length L_{g} ranges from about 1.0 mm to about 2.0 mm.

16. The lens assembly of claim 12 wherein the second diameter ranges from about 125 μm to about 250 μm.

17. The lens assembly of claim 12 wherein a reflective coating is disposed on the beam directing surface.

18. The lens assembly of claim 12, wherein at least a portion of the optical fiber and the rod are disposed in the bore.

19. A method of collecting interferometric data from a sample comprising:
transmitting light along an optical fiber such that the light crosses an interface between an optical fiber and a unitary rod comprising a spatially varying index of refraction gradient;
focusing light received by the unitary rod with a beam forming surface of the unitary rod on the sample;
forming a spot on the sample that ranges from about 5 μm to about 15 μm at a focal length that ranges from about 0.8 mm to about 2.0 mm and
detecting light reflected back from the sample through the optical fiber.

20. The method of claim 19 wherein a diameter of the unitary rod is greater than about 125 μm and less than about 250 μm.
